(19) United States
(12) Patent Application Publication
(10) Pub. No.: US 2024/0240217 A1
(43) Pub. Date: Jul. 18, 2024

(54) NUCLEOSIDES AND NUCLEOTIDES WITH 3' BLOCKING GROUPS AND CLEAVABLE LINKERS

(71) Applicant: Illumina, Inc., San Diego, CA (US)

(72) Inventors: Jean-Alexandre Richard, Singapore (SG); Xiangyuan Yang, Singapore (SG); Daniel Hartoyo Lukamto, Singapore (SG); Ramesh Neelakandan, Singapore (SG)

(21) Appl. No.: 18/530,865

(22) Filed: Dec. 6, 2023

Related U.S. Application Data
(60) Provisional application No. 63/386,765, filed on Dec. 9, 2022.

(51) Int. Cl. C12P 19/34; C07H 15/26; C07H 19/048; C12Q 1/6869; G01N 21/64

(52) U.S. Cl. CPC C12P 19/34; C07H 15/26; C07H 19/048; C12Q 1/6869; G01N 21/6428; G01N 2021/6439

(57) ABSTRACT

Embodiments of the present disclosure relate to nucleotide and nucleoside molecules with 3′ vinyl or isonitrile containing blocking groups and/or tetrazine or strained unsaturated ring containing cleavable linkers. Additionally, the present disclosure provides methods of using the nucleoside/nucleotide in oligonucleotide synthesis, and methods of sequencing using the nucleotide described herein.

### CROSS-REFERENCE TO RELATED APPLICATIONS

The present application claims the benefit of priority to U.S. Provisional Application No. 63/386,765, filed Dec. 9, 2022, the content of which is incorporated by reference in its entirety.

### FIELD

The present disclosure generally relates to nucleotides, nucleosides, or oligonucleotides comprising 3′ blocking groups and cleavable linkers. Methods of using the nucleotides, nucleosides, or oligonucleotides comprising the 3′ blocking groups and cleavable linkers are also disclosed.

### BACKGROUND

Advances in the study of molecules have been led, in part, by improvement in technologies used to characterize the molecules or their biological reactions. In particular, the study of the nucleic acids DNA and RNA has benefited from developing technologies used for sequence analysis and the study of hybridization events.

An example of the technologies that have improved the study of nucleic acids is the development of fabricated arrays of immobilized nucleic acids. These arrays consist typically of a high-density matrix of polynucleotides immobilized onto a solid support material. See, e.g., Jacobs et al., Combinatorial chemistry-applications of light-directed chemical synthesis, Trends Biotech. 12: 19-26 (1994), which describes ways of assembling the nucleic acids using a chemically sensitized glass surface protected by a mask, but exposed at defined areas to allow attachment of suitably modified nucleotide phosphoramidites. Fabricated arrays can also be manufactured by the technique of “spotting” known polynucleotides onto a solid support at predetermined positions (e.g., Stimpson et al., real-time detection of DNA hybridization and melting on oligonucleotide arrays by using optical wave guides, Proc. Natl. Acad. Sci. 92: 6379-83 (1995)).

One way of determining the nucleotide sequence of a nucleic acid bound to an array is called “sequencing by synthesis” or “SBS”. This technique for determining the sequence of DNA ideally requires the controlled (i.e., one at a time) incorporation of the correct complementary nucleotide opposite the nucleic acid being sequenced. This allows for accurate sequencing by adding nucleotides in multiple cycles as each nucleotide residue is sequenced one at a time, thus preventing an uncontrolled series of incorporations from occurring. The incorporated nucleotide is read using an appropriate label attached thereto before removal of the label moiety and the subsequent next round of sequencing.

Sequencing technologies require protecting group strategies, linkers, and chemical means to trigger desired events in a timely manner. In order to ensure that only a single fluorescent label is read at a time each label must be chemically cleaved and subsequently washed away from the incorporated nucleotide. Such a reaction must be highly efficient with a favorable reaction equilibrium that will not interfere with previously incorporated nucleotides. Further, it is desirable that the fluorescent label be cleaved without significant chemical “scarring” to the incorporated nucleotide.

Structural modifications (“protecting group” or “blocking group”) may also be employed in order to ensure that only a single nucleotide is added to a growing nucleotide chain. After the nucleotide with the protecting group has been added, the protecting group is then removed, under reaction conditions which do not interfere with the integrity of the DNA being sequenced. The sequencing cycle can then continue with the incorporation of the next protected, labeled nucleotide.

There are many limitations on the types of 3′ blocking groups that can be added onto a nucleotide and still be suitable. The protecting group should prevent additional nucleotide molecules from being added to the polynucleotide chain whilst simultaneously being easily removable from the sugar moiety without causing damage to the polynucleotide chain. Furthermore, the modified nucleotide needs to be compatible with the polymerase or another appropriate enzyme used to incorporate it into the polynucleotide chain. The ideal protecting group must therefore exhibit long-term stability, be efficiently incorporated by the polymerase enzyme, cause blocking of secondary or further nucleotide incorporation, and have the ability to be removed under mild conditions that do not cause damage to the polynucleotide structure, preferably under aqueous conditions.

Reversible protecting groups have been described previously. For example, Metzker et al., (Nucleic Acids Research, 22 (20): 4259-4267, 1994) discloses the synthesis and use of eight 3′-modified 2-deoxyribonucleoside 5′-triphosphates (3′-modified dNTPs) and testing in two DNA template assays for incorporation activity. WO 2002/029003 describes a sequencing method which may include the use of an allyl protecting group to cap the 3′—OH group on a growing strand of DNA in a polymerase reaction. In addition, the development of a number of reversible protecting groups and methods of deprotecting them under DNA compatible conditions was previously reported in International Application Publication Nos. WO 2004/018497 and WO 2014/139596, each of which is hereby incorporated by reference in its entirety. In addition, cleavable linkers between the nucleotide and the fluorescent label are also described in WO2004/018493.

It is desirable to continue developing alternative 3′ blocking group and cleavable linkers that are highly efficient and interfere minimally with the resulting structure of the incorporated nucleotide. It is also desirable that the cleavage of the blocking group and the fluorescent linker be performed in tandem or in the same cleavage reaction.

### SUMMARY

One aspect of the present disclosure relates to a nucleoside or nucleotide having the structure of Formula (I):

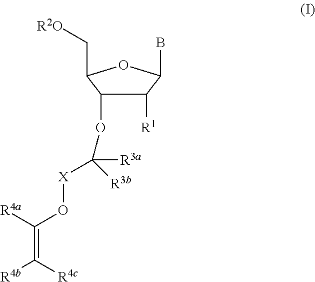

or a salt thereof, wherein:
B comprises a nucleobase;
R¹ is H, hydroxy, halo, unsubstituted or substituted C₁-C₆ alkoxy, or a hydroxy protecting group;
R² is H, a hydroxy protecting group, or —OR² is a monophosphate, diphosphate, triphosphate or phosphorothioate;
X is an unsubstituted or substituted C₆-C₁₀ arylene, or an unsubstituted or substituted 5 to 10 membered heteroarylene;
each of R^{3a} and R^{3b} is independently H, azido, halo, nitro, cyano, unsubstituted or substituted C₁-C₆ alkyl, C₁-C₆ haloalkyl, C₁-C₆ alkoxy, or C₁-C₆ haloalkoxy;
each of R^{4a}, R^{4b} and R^{4c} is independently H, halo, C₁-C₃ alkyl, C₁-C₃ haloalkyl, C₁-C₃ hydroxyalkyl, azido, cyano, unsubstituted or substituted phenyl, unsubstituted or substituted 5 to 6 membered heteroaryl, unsubstituted or substituted C₃-C₇ cycloalkyl, or unsubstituted or substituted 3 to 7 membered heterocyclyl. In some embodiments, the nucleoside or nucleotide has the structure of Formula (Ia):

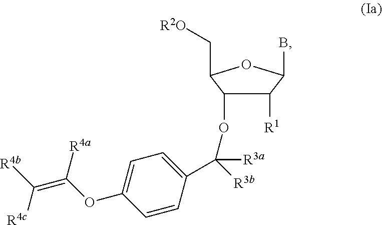

or a salt thereof.

Another aspect of the present disclosure relates to a method of determining the sequence of a target single-stranded polynucleotide, comprising: (a) incorporating a nucleotide of Formula (I) or (Ia) as described herein into a copy polynucleotide strand complementary to at least a portion of the target polynucleotide strand; (b) performing one or more fluorescent measurements for determining the identity of the nucleotide incorporated into the copy polynucleotide strand; and (c) removing the 3′ blocking group from the nucleotide incorporated into the copy polynucleotide strand.

One aspect of the present disclosure relates to a nucleoside or nucleotide comprising a nucleobase attached to a detectable label via a cleavable linker, wherein the nucleoside or nucleotide comprises a ribose or 2′ deoxyribose moiety and a 3′ blocking group, and wherein the cleavable linker comprises an unsubstituted or substituted C₇-C₁₄ unsaturated carbocyclic ring or ring system or unsubstituted or substituted 7 to 14 membered unsaturated heterocyclic ring or ring system, each comprising at least one double bond or one triple bond in the ring or ring system. In some embodiments, the nucleoside or nucleotide has the structure of Formula (IIa) or (IIb):

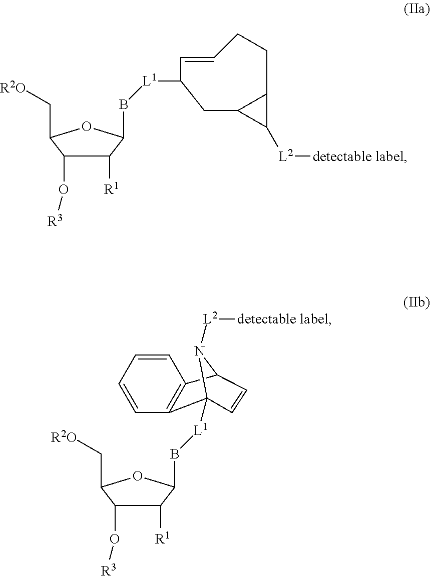

or a salt thereof, wherein
B is the nucleobase;
R¹ is H, hydroxy, halo, unsubstituted or substituted C₁-C₆ alkoxy, or a hydroxy protecting group;
R² is H, a hydroxy protecting group, or —OR² is a monophosphate, diphosphate, triphosphate or phosphorothioate;
R³ is a vinyl containing hydroxy blocking group; and
each of L¹ and L² is independently an optionally present linker moiety.

Another aspect of the present disclosure relates to a method of determining the sequence of a target single-stranded polynucleotide, comprising: (a) incorporating a nucleotide into a copy polynucleotide strand complementary to at least a portion of the target polynucleotide strand, wherein the nucleotide comprises a cleavable linker having an unsubstituted or substituted C₇-C₁₄ unsaturated carbocyclic ring or ring system or unsubstituted or substituted 7 to 14 membered unsaturated heterocyclic ring or ring system, each comprising at least one double bond or one triple bond in the ring or ring system (for example, a nucleotide of Formula (IIa) or (IIb)); (b) performing one or more fluorescent measurements for determining the identity of the nucleotide incorporated into the copy polynucleotide strand; and (c) removing the detectable label from the nucleotide incorporated into the copy polynucleotide strand.

One aspect of the present disclosure relates to a nucleoside or nucleotide comprising a nucleobase attached to a detectable label via a cleavable linker, wherein the nucleoside or nucleotide comprises a ribose or 2′ deoxyribose moiety and a 3′ blocking group, and wherein the cleavable linker comprises an unsubstituted or substituted tetrazine moiety. In some embodiments, the nucleoside or nucleotide has the structure of Formula (III):

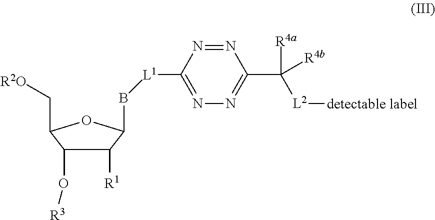

or a salt thereof, wherein
B is the nucleobase;
R¹ is H, hydroxy, halo, unsubstituted or substituted C₁-C₆ alkoxy, or a hydroxy protecting group;
R² is H, a hydroxy protecting group, or —OR² is a monophosphate, diphosphate, triphosphate or phosphorothioate;
R³ is a vinyl containing hydroxy blocking group; and
each of R^{3a} and R^{3b} is independently H, azido, halo, nitro, cyano, unsubstituted or substituted C₁-C₆ alkyl, C₁-C₆ haloalkyl, C₁-C₆ alkoxy, or C₁-C₆ haloalkoxy; and
each of L¹ and L² is independently an optionally present linker moiety.

Another aspect of the present disclosure relates to a method of determining the sequence of a target single-stranded polynucleotide, comprising: (a) incorporating a nucleotide into a copy polynucleotide strand complementary to at least a portion of the target polynucleotide strand, wherein the nucleotide comprises a 2′ deoxyribose moiety and a 3′ blocking group, and a nucleobase attached to a detectable label via a cleavable linker having an unsubstituted or substituted tetrazine moiety (for example, a nucleotide of Formula (III)); (b) performing one or more fluorescent measurements for determining the identity of the nucleotide incorporated into the copy polynucleotide strand; and (c) removing the detectable label from the nucleotide incorporated into the copy polynucleotide strand.

One aspect of the present disclosure relates to a nucleoside or nucleotide comprising a ribose or 2′ deoxyribose moiety and a 3′ blocking group, and a detectable label attached to the 3′ blocking group optionally via a linker, wherein the 3′ blocking group comprises an isonitrile moiety. In some embodiments, the nucleoside or nucleotide has the structure of Formula (IV):

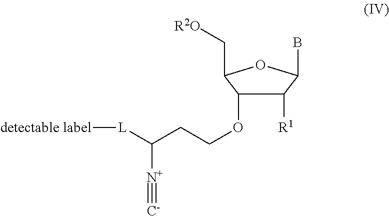

or a salt thereof, wherein:
B is a nucleobase;
R¹ is H, hydroxy, halo, unsubstituted or substituted C₁-C₆ alkoxy, or a hydroxy protecting group;
R² is H, a hydroxy protecting group, or —OR³ is a monophosphate, diphosphate, triphosphate or phosphorothioate; and
L is an optionally present linker moiety.

Another aspect of the present disclosure relate to a method of determining the sequence of a target single-stranded polynucleotide, comprising: (a) incorporating a nucleotide into a copy polynucleotide strand complementary to at least a portion of the target polynucleotide strand, wherein the nucleotide comprises a 2′ deoxyribose moiety and a 3′ blocking group, and a detectable label attached to the 3′ blocking group optionally via a linker, wherein the 3′ blocking group comprises an isonitrile moiety (for example, a nucleotide of Formula (IV)); (b) performing one or more fluorescent measurements for determining the identity of the nucleotide incorporated into the copy polynucleotide strand; and (c) removing the 3′ blocking group from the nucleotide incorporated into the copy polynucleotide strand.

Additional aspect of the present disclosure relates to an oligonucleotide or polynucleotide comprising the nucleotide described herein. For example, a nucleotide of Formula (I) or (Ia)); a nucleotide with a cleavable linker comprises an unsubstituted or substituted C₇-C₁₄ unsaturated carbocyclic ring or ring system or unsubstituted or substituted 7 to 14 membered unsaturated heterocyclic ring or ring system, each comprising at least one double bond or one triple bond in the ring or ring system (e.g., a nucleotide of Formula (IIa) or (IIb)); a nucleotide with a 3′ blocking group comprising an unsubstituted or substituted tetrazine moiety (e.g., the nucleotide of Formula (III)); or the nucleotide with a 3′ blocking group comprising an isonitrile moiety (e.g., the nucleotide of Formula (IV)).

Additional aspect of the present disclosure relates to a method of controlled synthesis of an oligonucleotide or polynucleotide, comprising:
contacting a nucleotide described herein with the oligonucleotide or polynucleotide in the presence of a polymerase; and
incorporating the nucleotide to the 3′ end of the oligonucleotide or polynucleotide.

### DETAILED DESCRIPTION

Embodiments of the present disclosure relate to nucleosides and nucleotides with 3′ blocking groups and cleavable linkers. According to various embodiments of the present disclosure a cleavage reaction of a 3′ blocking group or a cleavable linker may be performed via chemical reactions that are high yielding, proceed rapidly and selectively under desirable conditions, and create minimal byproducts.

Various reactions of the present embodiment enable the cleavage of a 3′ blocking group or a linker moiety via a click chemistry reaction. Such reactions, in accordance with various embodiments, enable the removal of the 3′ protecting group or the linker moiety with minimal residue on the incorporated nucleotide. These reactions which leave minimal residue or scarring on the incorporated nucleotide may be termed “scarless” or “quasi-scarless” reactions.

Embodiments of the present disclosure may also relate to sequencing applications, for example, sequencing-by-synthesis (SBS) that utilizes one or more nucleotide described herein. In some embodiments the nucleotide comprises a fluorescent label that is covalently attached to the nucleotide via a cleavable linker at a position different from the 3′ blocking group. In other embodiments, the nucleotide comprises a label covalently attached to (optionally through a cleavable linker) a 3′ blocking group moiety that allows for cleavage of the 3′ blocking group and the label in a single step of reaction. The 3′ blocking groups offer improved stability during the synthesis of the fully functionalized nucleotides (ffNs) and also great stability in solution during formulation, storage and operation on the sequencing instruments. In addition, the 3′ blocking groups described herein may also achieve low pre-phasing, lower signal decay for improved data quality, which enables longer reads from the sequencing applications.

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffN Fully functionalized nucleotide
RT Room temperature
SBS Sequencing by Synthesis

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, any “R” group(s) represent substituents that can be attached to the indicated atom. An R group may be substituted or unsubstituted. If two “R” groups are described as “together with the atoms to which they are attached” forming a ring or ring system, it means that the collective unit of the atoms, intervening bonds and the two R groups are the recited ring. For example, when the following substructure is present:

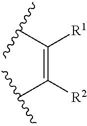

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

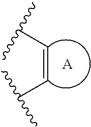

where A is an aryl ring or a carbocyclyl containing the depicted double bond.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 6 carbon atoms. The heteroalkyl group may be designated as “C₁-C₆ heteroalkyl” or similar designations. The heteroalkyl group may contain one or more heteroatoms. By way of example only, “C₄-C₆ heteroalkyl” indicates that there are four to six carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazolylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a C₂-C₈ alkoxyalkyl or (C₁-C₆ alkoxy)C₁-C₆ alkyl, for example, —(CH₂)₁₋₃—OCH₃.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆alkyl, C₁-C₆ alkoxy, C₁-C₆ haloalkyl, and C₁-C₆ haloalkoxy), halo, —CN, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfino, —OSO₂C₁₋₄alkyl, and oxo (═O). Wherever a group is described as “optionally substituted” that group can be substituted with the above substituents.

As used herein, the term “hydroxy” refers to a —OH group.

As used herein, the term “cyano” group refers to a “—CN” group.

As used herein, the term “azido” refers to a —N₃ group.

As used herein, the term “azidomethyl” refers to a “—CH₂N₃” group. In the context of a 3′ blocking group, the term “3′ azidomethyl” means that the azidomethyl group that is covalently attached to the 3′ oxygen of the ribose or deoxyribose ring of the nucleoside or nucleotide.

As used herein the term “vinyl” refers to a “—CH═CH₂”. In the context of a 3′ blocking group, the term “3′ vinyl” means that the vinyl group that is covalently attached to the 3′ oxygen of the ribose or deoxyribose ring of the nucleoside or nucleotide or the vinyl group is attached to the 3′ oxygen of the ribose or deoxyribose ring of the nucleoside or nucleotide through a cleavable linker.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxyl group that is present in ribose. The nitrogen containing heterocyclic base can be purine or pyrimidine base. Purine bases include adenine (A) and guanine (G), and modified derivatives or analogs thereof, such as deazapurine or 7-deazapurine (e.g., deaza adenine, 7-deaza adenine, deaza guanine, or 7-deaza guanine). Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, deazapurine, adenine, deaza adenine, guanine, deaza guanine, 7-deaza adenine, 7-deaza guanine, hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, “derivative” or “analogue” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Antisense oligonucleotides: a new therapeutic principle, Chemical Reviews 90:543-84 (1990). Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl (Bn); substituted benzyl; alkylcarbonyls (e.g., t-butoxycarbonyl (BOC), acetyl (i.e., —C(═O)CH₃ or Ac), or isobutyryl (iBu); arylalkylcarbonyls (e.g., benzyloxycarbonyl or benzoyl (i.e., —C(═O)Ph or Bz)); substituted methyl ether (e.g., methoxymethyl ether (MOM)); substituted ethyl ether (e.g., methoxyethyl ether (MOE); a substituted benzyl ether; tetrahydropyranyl ether; silyl ethers (e.g., trimethylsilyl (TMS), triethylsilyl, triisopropylsilyl, t-butyldimethylsilyl (TBDMS), tri-iso-propylsilyloxymethyl (TOM), or t-butyldiphenylsilyl); esters (e.g., benzoate ester); carbonates (e.g., methoxymethylcarbonate); sulfonates (e.g., tosylate or mesylate); acyclic ketal (e.g., dimethyl acetal); cyclic ketals (e.g., 1,3-dioxane or 1,3-dioxolanes); acyclic acetal; cyclic acetal; acyclic hemiacetal; cyclic hemiacetal; cyclic dithioketals (e.g., 1,3-dithiane or 1,3-dithiolane); and triarylmethyl groups (e.g., trityl; monomethoxytrityl (MMTr); 4,4′-dimethoxytrityl (DMTr); or 4,4′,4″-trimethoxytrityl (TMTr)).

Examples of hydroxyl protecting groups include without limitation, acetyl, t-butyl, t-butoxymethyl, methoxymethyl, tetrahydropyranyl, 1-ethoxyethyl, 1-(2-chloroethoxy)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoro-acetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMTr), trimethoxytrityl, 1(2-fluorophenyl)-4-methoxypiperidin-4-yl (FPMP), 9-phenylxanthen-9-yl (Pixyl) and 9-(p-methoxyphenyl)xanthen-9-yl (MOX), wherein more commonly used hydroxyl protecting groups include without limitation, benzyl, 2,6-dichlorobenzyl, t-butyldimethylsilyl, t-butyldiphenylsilyl, benzoyl, mesylate, tosylate, dimethoxytrityl (DMTr), 9-phenylxanthine-9-yl (Pixyl) and 9-(p-methoxyphenyl)xanthine-9-yl (MOX).

As used herein, the term “phosphate” is used in its ordinary sense as understood by those skilled in the art, and includes its protonated forms (for example,

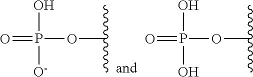 [SMILES: *OP(=O)([O-])O ; *OP(=O)(O)O]

). As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably.

Nucleosides or Nucleotides with 3′ Blocking Groups and Cleavable Linkers

### A. Nucleosides/Nucleotides of Formula (I)

One aspect of the present disclosure relates to a nucleoside or nucleotide having the structure of Formula (I):

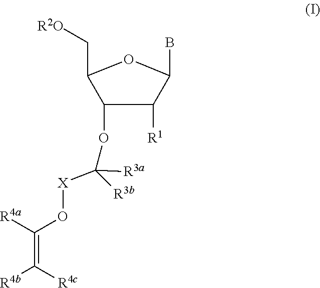

or a salt thereof, wherein:
B comprises a nucleobase;
R¹ is H, hydroxy, halo, unsubstituted or substituted C₁-C₆ alkoxy, or a hydroxy protecting group;
R² is H, a hydroxy protecting group, or —OR² is a monophosphate, diphosphate, triphosphate or phosphorothioate;
X is an unsubstituted or substituted C₆-C₁₀ arylene, or an unsubstituted or substituted 5 to 10 membered heteroarylene;
each of R^{3a} and R^{3b} is independently H, azido, halo, nitro, cyano, unsubstituted or substituted C₁-C₆ alkyl, C₁-C₆ haloalkyl, C₁-C₆ alkoxy, or C₁-C₆ haloalkoxy;
each of R^{4a}, R^{4b} and R^{4c} is independently H, halo, C₁-C₃ alkyl, C₁-C₃ haloalkyl, C₁-C₃ hydroxyalkyl, azido, cyano, unsubstituted or substituted phenyl, unsubstituted or substituted 5 to 6 membered heteroaryl, unsubstituted or substituted C₃-C₇ cycloalkyl, or unsubstituted or substituted 3 to 7 membered heterocyclyl.

In some embodiments, X is phenylene, and the nucleoside or nucleotide of Formula (I) has a structure of Formula (Ia):

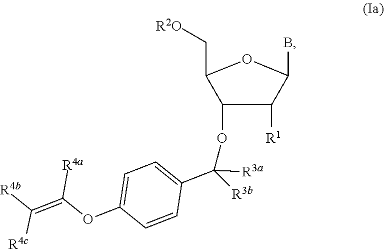

or a salt thereof.

In some embodiments of the nucleoside/nucleotide of Formula (I) or Formula (Ia), at least one of R^{4a}, R^{4b} and R^{4c} is methyl, ethyl, n-propyl, isopropyl, fluoro, chloro, —CHF₂, —CH₂F, —CH₂Cl, —CHCl₂, or —CF₃. In one embodiment, each of R^{4a}, R^{4b} and R^{4c} is H. In other embodiments, at least one of R^{4b} and R^{4c} is fluoro. In some further embodiments, each of R^{3a} and R^{3b} is H. In some embodiments, R¹ is H, hydroxy, or a hydroxy protecting group selected from the group consisting of

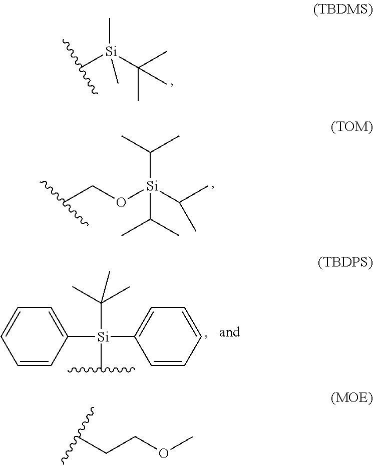

that covalently attached to the 2′ oxygen atom. In other embodiments, R¹ is —O-2′-bis(2-acetoxyethoxy)methyl,

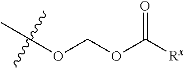

(wherein R^{x} is an optionally substituted C₆-C₁₀ aryl, an optionally substituted 5 to 10-membered heteroaryl, or optionally substituted 5 to 10-membered heterocyclyl),

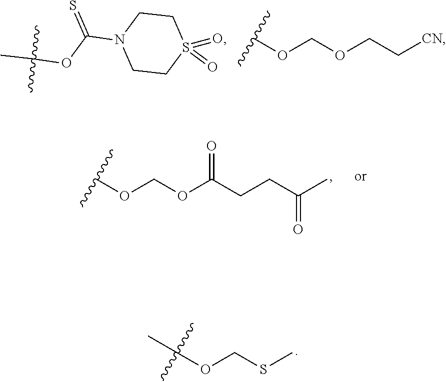

In one embodiment, R¹ is H. In another embodiment, R¹ is fluoro. In another embodiment, R¹ is methoxy or ethoxy. In some embodiments, B comprises a nucleobase covalently bounded to a detectable label, optionally through a linker. In further embodiments, B is

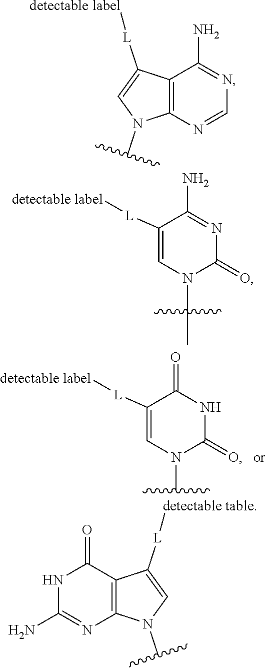

In some embodiments, the nucleoside or nucleotide is nucleotide having 2′ deoxyribose, and —OR² is triphosphate. Further embodiments relate to an oligonucleotide or polynucleotide comprising a nucleotide of Formula (I) or (Ia) as described herein.
B. Nucleosides/Nucleotides with Strained Unsaturated Cyclic Ring Containing Cleavable Linker

Some embodiments of the present disclosure relate to a nucleoside or nucleotide comprising a nucleobase attached to a detectable label via a cleavable linker, wherein the nucleoside or nucleotide comprises a ribose or 2′ deoxyribose moiety and a 3′ blocking group, and wherein the cleavable linker comprises an unsubstituted or substituted C₇-C₁₄ unsaturated carbocyclic ring or ring system or unsubstituted or substituted 7 to 14 membered unsaturated heterocyclic ring or ring system, each comprising at least one double bond or one triple bond in the ring or ring system. In some embodiments, the C₇-C₁₄ unsaturated carbocyclic ring or ring system comprises a trans-cyclooctene moiety. In some embodiments, the 7 to 14 membered heterocyclic ring or ring system comprises a 7-azanorbornene moiety. In some further embodiments, the 3′ blocking group comprises a vinyl moiety. In some embodiments, the nucleoside or nucleotide has the structure of Formula (IIa) or (IIb):

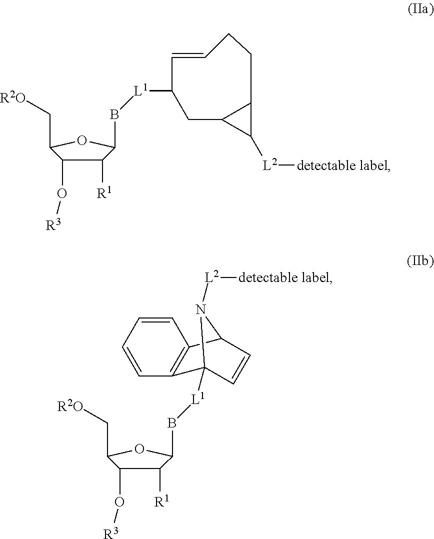

or a salt thereof, wherein
B is the nucleobase;
R¹ is H, hydroxy, halo, unsubstituted or substituted C₁-C₆ alkoxy, or a hydroxy protecting group;
R² is H, a hydroxy protecting group, or —OR² is a monophosphate, diphosphate, triphosphate or phosphorothioate;
R³ is a vinyl containing hydroxy blocking group; and
each of L¹ and L² is independently an optionally present linker moiety.

In some embodiments of the nucleoside/nucleotide of Formula (IIa) or Formula (IIb), R³ is

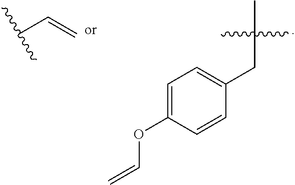

In some embodiments, R¹ is H, hydroxy, or a hydroxy protecting group selected from the group consisting of

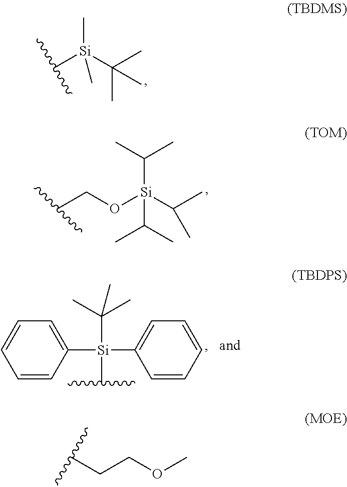

that covalently attached to the 2′ oxygen atom. In other embodiments, R¹ is —O-2′-bis(2-acetoxyethoxy)methyl,

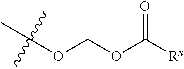

(wherein R an optionally substituted C₆-C₁₀ aryl, an optionally substituted 5 to 10-membered heteroaryl, or optionally substituted 5 to 10-membered heterocyclyl),

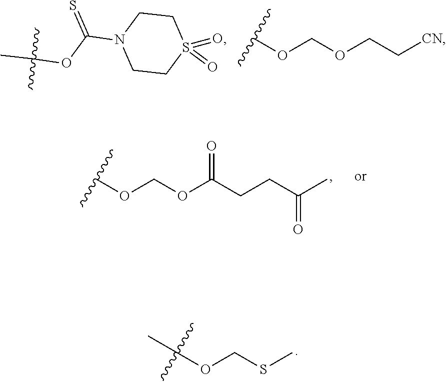

In one embodiment, R¹ is H. In another embodiment, R¹ is fluoro. In yet another embodiment, R¹ is methoxy. In further embodiments, B is

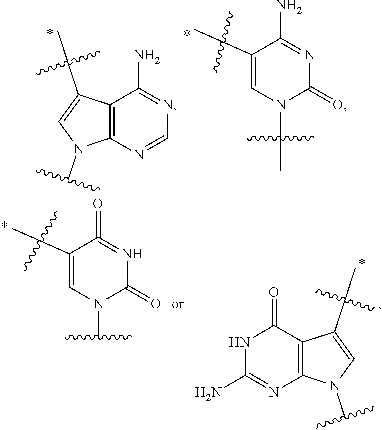

wherein * indicates the point of attachment of the nucleobase to L¹. In additional embodiments, L¹ comprises

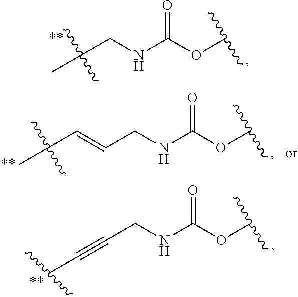

wherein ** indicates the point of attachment of L¹ to the nucleobase. In further embodiments, the nucleoside or nucleotide is nucleotide triphosphate having 2′ deoxyribose. Further embodiments relate to an oligonucleotide or polynucleotide comprising a nucleotide disclosed herein.
C. Nucleosides/Nucleotides with Tetrazine Containing Cleavable Linker

Additional embodiments of the present disclosure relate to a nucleoside or nucleotide comprising a nucleobase attached to a detectable label via a cleavable linker, wherein the nucleoside or nucleotide comprises a ribose or 2′ deoxyribose moiety and a 3′ blocking group, and wherein the cleavable linker comprises an unsubstituted or substituted tetrazine moiety. In some such embodiments, the nucleoside or nucleotide has the structure of Formula (III):

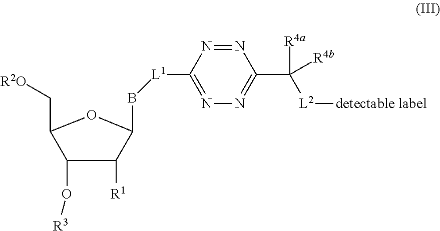

or a salt thereof, wherein
B is the nucleobase;
R¹ is H, hydroxy, halo, unsubstituted or substituted C₁-C₆ alkoxy, or a hydroxy protecting group;
R² is H, a hydroxy protecting group, or —OR² is a monophosphate, diphosphate, triphosphate or phosphorothioate;
R³ is a vinyl containing hydroxy blocking group; and
each of R^{3a} and R^{3b} is independently H, azido, halo, nitro, cyano, unsubstituted or substituted C₁-C₆ alkyl, C₁-C₆ haloalkyl, C₁-C₆ alkoxy, or C₁-C₆ haloalkoxy; and
each of L¹ and L² is independently an optionally present linker moiety.

In some embodiments of the nucleoside/nucleotide of Formula (III), R¹ is H, hydroxy, or a hydroxy protecting group selected from the group consisting of

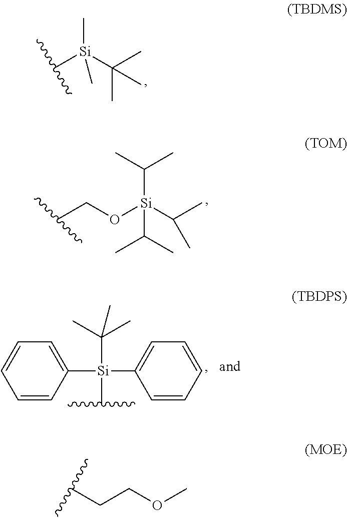

that covalently attached to the 2′ oxygen atom. In other embodiments, R¹ is —O-2′-bis(2-acetoxyethoxy)methyl,

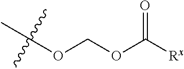

(wherein R^{x} is an optionally substituted C₆-C₁₀ aryl, an optionally substituted 5 to 10-membered heteroaryl, or optionally substituted 5 to 10-membered heterocyclyl),

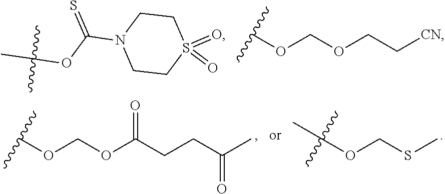

In one embodiment, R¹ is H. In another embodiment, R¹ is fluoro. In yet another embodiment, R¹ is methoxy. In some embodiments, B is

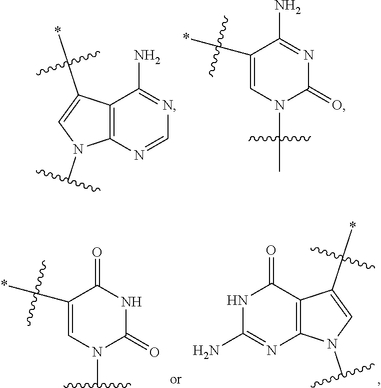

wherein * indicates the point of attachment of the nucleobase to L¹. In further embodiments of Formula (III), each of R^{4a} and R^{4b} is H. In further embodiments of Formula (III), L¹ comprises

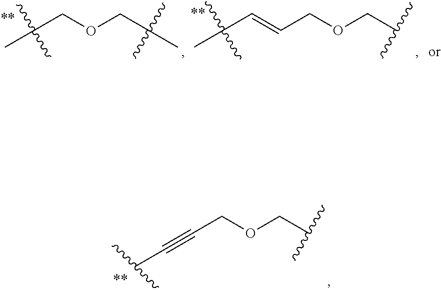 [SMILES: *COC* ; */C=C/COC* ; *C#CCOC*]

wherein ** indicates the point of attachment of L¹ to the nucleobase. In further embodiments the nucleoside or nucleotide is nucleotide triphosphate having 2′ deoxyribose. Further embodiments of the present disclosure relate to an oligonucleotide or polynucleotide comprising a nucleotide disclosed herein.
D. Nucleosides/Nucleotides with Isonitrile Containing 3′ Blocking Group

Additional embodiments of the present disclosure relate to a nucleoside or nucleotide comprising a ribose or 2′ deoxyribose moiety and a 3′ blocking group, and a detectable label attached to the 3′ blocking group optionally via a linker, wherein the 3′ blocking group comprises an isonitrile moiety. In additional embodiments, the nucleoside or nucleotide has a structure of Formula (IV):

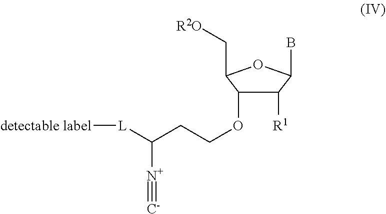

or a salt thereof, wherein:
B is a nucleobase;
R¹ is H, hydroxy, halo, unsubstituted or substituted C₁-C₆ alkoxy, or a hydroxy protecting group;
R² is H, a hydroxy protecting group, or —OR³ is a monophosphate, diphosphate, triphosphate or phosphorothioate; and
L is an optionally present linker moiety.

In some embodiments the nucleoside/nucleotide of Formula (IV), R¹ is H, hydroxy, or a hydroxy protecting group selected from the group consisting of

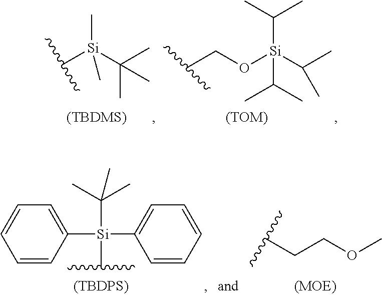

that covalently attached to the 2′ oxygen atom. In other embodiments, R¹ is —O-2′-bis(2-acetoxyethoxy)methyl,

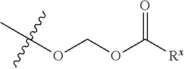

wherein R^{x} is an optionally substituted C₆-C₁₀ aryl, an optionally substituted 5 to 10-membered heteroaryl, or optionally substituted 5 to 10-membered heterocyclyl),

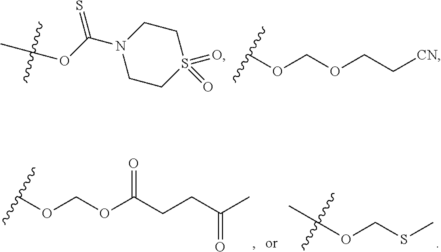

In one embodiment, R¹ is H. In another embodiment, R¹ is fluoro. In yet another embodiment, R¹ is methoxy. In some embodiments, B is

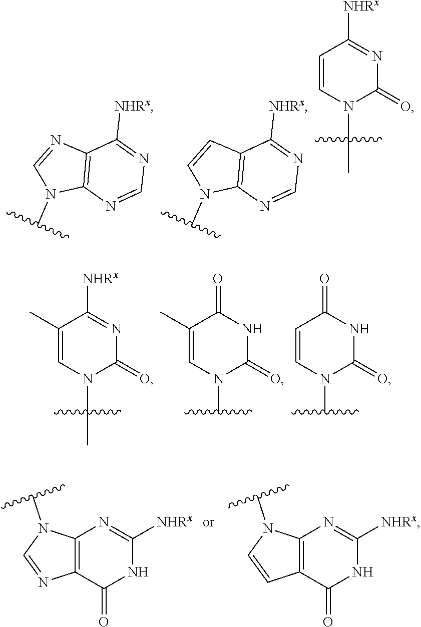

wherein R^{x} is H or an amino protecting group, or the hydrogen in —NHR^{x} is absent and R^{x} is a divalent amino protecting group. In some embodiments, R^{x} is H, —C(═O)C₁₋₆ alkyl or —C(═O)-phenyl. In some embodiments, the nucleoside or nucleotide is nucleotide triphosphate having 2′ deoxyribose. Further embodiments relate to an oligonucleotide or polynucleotide comprising a nucleotide disclosed herein.

### 3′ Vinyl Blocking Groups

Oligonucleotide synthesis encompasses both chemical and biochemical methods of manufacturing an oligonucleotide. Illumina's suite of products includes both short and long oligo/polymers of DNA/RNA, which may be synthesized chemically using nucleoside phosphoramidites on solid supports, and/or biochemically using polymerases/ligases and nucleoside triphosphates. To achieve these methods of oligonucleotide synthesis, a protecting group strategy may be helpful to ensure clean, efficient and robust productions.

Some embodiments disclosed herein relate to novel protecting group strategies for use in oligonucleotide synthesis. The synthetic or semi-synthetic manipulation of nucleosides in oligonucleotide synthesis may involve a protecting group strategy which includes a series of incorporation stages followed by deprotection stages. One way to achieve this is to place stable and tolerant blocking group on the 3′ hydroxy of the nucleosides/nucleotides. Furthermore, it may be helpful if the blocking groups themselves are tolerant to the oligonucleotide synthesis conditions. It may additionally be helpful if the deprotection stages and subsequent nucleotide steps are efficient and reliable.

Certain blocking groups have been previously described. For example, azidomethyl (AZM) has been used for biochemical synthesis with polymerases, and acid-labile groups such as 4,4′-dimethoxytrityl (DMT) has been used for solid-phase chemical synthesis. However, these groups may be too unstable for long-term storage and/or require the use of acid in production, which introduces side-reactions such as depurination, and may be detrimental to the synthesis of long oligonucleotides and genes. Furthermore, a more tolerant protecting group may allow orthogonal functional group tolerability.

Thus, a robust and tolerant protecting group with clean and reliable deprotection mechanism, while utilizing neutral pH conditions, may address some of the issues of previous blocking groups. One example strategy having fast and clean deprotection is the use of vinyl ethers as hydroxyl protecting groups. These groups are discussed in greater detail within Voronin et al., Examining the vinyl moiety as a protecting group for hydroxyl (—OH) functionality under basic conditions, Organic Chem. Frontiers, 2020, 7, 1334-42, incorporated herein by reference. Furthermore, a vinyl group is also acid labile.

Alcohols may be protected as either ethers or esters, as discussed by P. G. W. Wuts et al., Greene's Protective Groups in Organic Synthesis, John Wiley & Sons, Inc., Hoboken, NJ, 4 edn., (2006), incorporated herein by reference. Of ethers and esters, ethers may be more tolerant to strong reactants including oxidizing agents and organometallic compounds. However, this property may also make ethers more difficult to deprotect. Furthermore, the use of ethers as protecting groups may involve expensive, harsh or hazardous reagents, strongly acidic conditions, long reaction times and tedious workup procedures. For instance, although alkyl and benzyl ethers are highly stable, their deprotections may involve hazardous reagents, such as BBr₃, BF₃·Et₂O, HgCl₂, 2,3-dichloro-5,6-dicyano-p-benzoquinone (DDQ), alkaline metals, or potentially explosive hydrogenation conditions. Acid-labile silyl ethers can be removed under mild conditions. However, the cost of silyl chlorides needed for the protection may be high and may introduce bulky substituents that interfere with polymerase activities in the biochemical synthesis of oligonucleotides. The simpler methoxymethyl (MOM) ethers may be used but the removal of MOM blocking group require rather harsh reaction conditions.

Alternatively, vinyl ethers may be a viable option to provide higher stability while allowing deprotection with safe and readily-available tetrazine derivatives. The use of tetrazine derivatives is discussed further within B. L. Oliveira et al., Inverse electron demand Diels-Alder reactions in chemical biology, Bernardes. Chem. Soc. Rev. 2017, 46, 4895-950, which is incorporated herein by reference. The deprotection of vinyl ethers with tetrazine may involve relatively fast kinetics, approaching a ‘click’-type reaction, which has been termed inverse electron-demand Diels-Alder (IEDDA), under relatively low concentrations, for example within the M range. The IEDDA is described in E. Jimenez-Moreno et al., Vinyl Ether/Tetrazine Pair for the Traceless Release of Alcohols in Cells, Angew. Chem. Int. Ed. 2017, 56, 243, which is incorporated herein by reference.

### Linkers

The detectable label as disclosed herein may include a reactive linker group at one of the substituent positions for covalent attachment of the detectable label to the nucleoside/nucleotide described herein. Reactive linking groups are moieties capable of forming a bond (e.g., a covalent or non-covalent bond), in particular a covalent bond. In a particular embodiment the linker may be a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO 2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

### Biosynthesis of Oligonucleotides

Oligonucleotides are frequently used in synthetic biology. One approach to the synthesis of oligonucleotides, is the enzymatic synthesis of oligonucleotides using template-independent polymerase. Such polymerases can incorporate nucleotides to an oligonucleotide strand without need for a template strand. Template-independent polymerase may include a nucleotidyl transferase (such as terminal deoxynucleotidyl transferase (TdT)), a PolyA polymerase, and/or a CCA-adding RNA polymerase. The template-independent polymerase may be specific to RNA and/or DNA. Stable, easily removed 3′-blocks may provide a greater level of control in oligonucleotide synthesis using a template-independent polymerase.

Some embodiments herein relate to a method of controlled synthesis of an oligonucleotide or polynucleotide, comprising: contacting a nucleotide described herein with the oligonucleotide or polynucleotide in the presence of a polymerase; and incorporating the nucleotide to the 3′ end of the oligonucleotide or polynucleotide. In some embodiments, the method further comprises removing the 3′ blocking group of the incorporated nucleotide to generate a 3′ hydroxy group on the incorporated nucleotide, and incorporating a second nucleotide.

In some embodiments, when the nucleotide incorporated is a nucleotide of Formula (I) or (Ia) as described herein, the removal of the 3′ blocking group of the incorporated nucleotide can be achieved with a tetrazine reagent. In some embodiments, when the tetrazine reagent is selected from the group consisting of

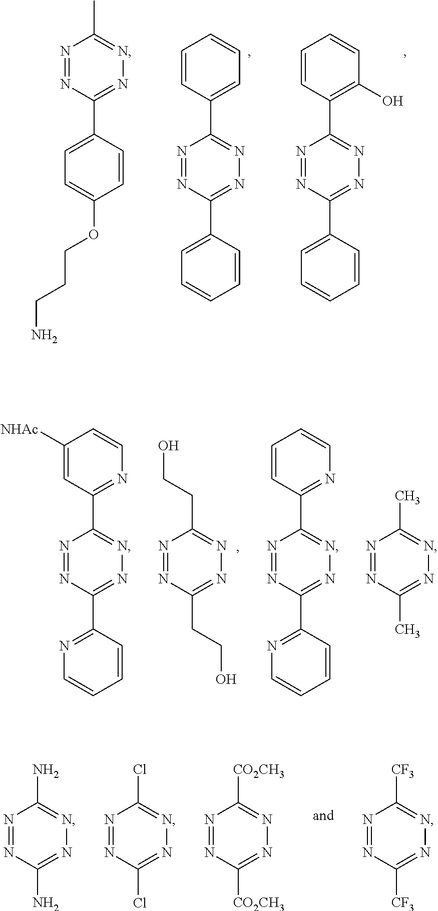

and substituted variants thereof.

In some other embodiments, when the nucleotide incorporated has a 3′ blocking group comprising an isonitrile moiety, such as the nucleotide of Formula (IV) as described herein, the removal of the 3′ blocking group of the incorporated nucleotide can be achieved with a tetrazine reagent described herein.

### Tetrazine as a Cleavage Reagent

Various tetrazine species may be used to deprotect 3′ vinyl blocking group described herein. The self-immolative cleavage mechanism may be similar for simple tetrazines, such as

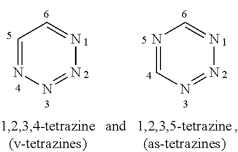 [SMILES: c1cnnnn1 ; c1ncnnn1]

and substituted tetrazines, such as

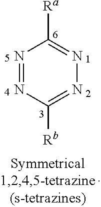

Thus, each of v-tetrazine, s-tetrazine, and/or as-tetrazine may be used in the self-immolative cleavage reaction. Furthermore, the R^{a} and R^{b} groups of s-tetrazine may be selected to tune the reactivity and rate of the deprotection step. Generally, electron-donating groups slow down the rate of reactions but improve stability, while electron-withdrawing groups improve the rate of reactions but reduce stability. In effect, there is a trade-off between stability and reactivity of 1,2,4,5-tetrazines in an IEDDA reaction which is modulated by electron donating or electron-withdrawing groups. Generally speaking, presence of electron donating groups contributes to the stability of the compound. Presence of electron withdrawing groups reduces the stability of the compound but may increase the speed of the deprotection reaction. For example, each R^{a} and R^{b} may be independently H, C₁-C₆ alkyl, substituted C₁-C₆ alkyl, C₁-C₆ hydroxyalkyl, C₁-C₆ haloalkyl, C₁-C₆ aminoalkyl, (C₁-C₆ alkoxy)C₁-C₆ alkyl, —O—(C₁-C₆ alkoxy)C₁-C₆ alkyl, amino, substituted amino, halo, cyano, nitro, carboxyl, C-carboxy (e.g., —C(O)OC₁-C₆ alkyl), C₁-C₆ alkoxy, substituted C₁-C₆ alkoxy, C₁-C₆ haloalkoxy, optionally substituted phenyl, optionally substituted 5 to 10 membered heteroaryl (e.g., pyridyl, pyrimidyl, etc.), optionally substituted C₃-C₇ cycloalkyl, or optionally substituted 3 to 10 membered heterocyclyl. In some instances, R^{a} and R^{b} are the same. In other cases, R^{a} and R^{b} are different.

### Kits

Also provided herein are kits including one or more nucleotides, wherein one type of nucleotide is a nucleotide described herein.

In some embodiments, the kit may contain four types of labeled nucleotides (A, C, G and T or U), where the first of the four nucleotides is a nucleotide described herein. In such a kit, each of the four nucleotides can be labeled with a compound that is the same or different from the label on the other three nucleotides. Alternatively, a first of the four nucleotides is a labeled nucleotide describe herein, a second of the four nucleotides carries a second label, a third nucleotide carries a third label, and a fourth nucleotide is unlabeled (dark). As another example, a first of the four nucleotides is a labeled nucleotide described herein, a second of the four nucleotides carries a second label, a third nucleotide carries a mixture of two labels, and a fourth nucleotide is unlabeled (dark). Thus, one or more of the label compounds can have a distinct absorbance maximum and/or emission maximum such that the compound(s) is(are) distinguishable from other compounds. For example, each compound can have a distinct absorbance maximum and/or emission maximum such that each of the compounds is spectrally distinguishable from the other three compounds (or two compounds if the fourth nucleotide is unlabeled). It will be understood that parts of the absorbance spectrum and/or emission spectrum other than the maxima can differ and these differences can be exploited to distinguish the compounds. The kit may be such that two or more of the compounds have a distinct absorbance maximum.

The nucleotides or kits that are set forth herein may be used to detect, measure, or identify a biological system (including, for example, processes or components thereof). Exemplary techniques that can employ the compounds, nucleotides or kits include sequencing, expression analysis, hybridization analysis, genetic analysis, RNA analysis, cellular assay (e.g., cell binding or cell function analysis), or protein assay (e.g., protein binding assay or protein activity assay). The use may be on an automated instrument for carrying out a particular technique, such as an automated sequencing instrument. The sequencing instrument may contain two light sources operating at different wavelengths.

In a particular embodiment, the labeled nucleotide(s) described herein may be supplied in combination with unlabeled or native nucleotides, or any combination thereof. Combinations of nucleotides may be provided as separate individual components (e.g., one nucleotide type per vessel or tube) or as nucleotide mixtures (e.g., two or more nucleotides mixed in the same vessel or tube).

Where kits comprise a plurality, particularly two, or three, or more particularly four, nucleotides, the different nucleotides may be labeled with different dye compounds, or one may be dark, with no dye compounds. Where the different nucleotides are labeled with different dye compounds, it is a feature of the kits that the dye compounds are spectrally distinguishable fluorescent dyes. As used herein, the term “spectrally distinguishable fluorescent dyes” refers to fluorescent dyes that emit fluorescent energy at wavelengths that can be distinguished by fluorescent detection equipment (for example, a commercial capillary-based DNA sequencing platform) when two or more such dyes are present in one sample. When two nucleotides labeled with fluorescent dye compounds are supplied in kit form, it is a feature of some embodiments that the spectrally distinguishable fluorescent dyes can be excited at the same wavelength, such as, for example by the same light source. When four nucleotides labeled with fluorescent dye compounds are supplied in kit form, it is a feature of some embodiments that two of the spectrally distinguishable fluorescent dyes can both be excited at one wavelength and the other two spectrally distinguishable dyes can both be excited at another wavelength. Particular excitation wavelengths for the dyes are between 450-460 nm, 490-500 nm, or 520 nm or above (e.g., 532 nm).

In addition to the labeled nucleotides, the kit may comprise together at least one additional component. The further component(s) may be one or more of the components identified in a method set forth herein or in the Examples section below. Some non-limiting examples of components that can be combined into a kit of the present disclosure are set forth below. In some embodiments, the kit further comprises a DNA polymerase (such as a mutant DNA polymerase) and one or more buffer compositions. Non-limiting examples of DNA polymerase may be used in the present disclosure include those disclosed in WO 2005/024010, US Publication Nos. 2020/0131484 A1 and 2020/0181587 A1, each of which is incorporated by reference herein in its entirety. One buffer composition may comprise antioxidants such as ascorbic acid or sodium ascorbate, which can be used to protect the dye compounds from photo damage during detection. Additional buffer composition may comprise a reagent can may be used to cleave the 3′ blocking group and/or the cleavable linker. For example, a water-soluble phosphines or water-soluble transition metal catalysts formed from a transition metal and at least partially water-soluble ligands, such as a palladium complex. Various components of the kit may be provided in a concentrated form to be diluted prior to use. In such embodiments a suitable dilution buffer may also be included. Again, one or more of the components identified in a method set forth herein can be included in a kit of the present disclosure. In any embodiments of the nucleotide or labeled nucleotide described herein, the nucleotide contains a 3′ blocking group.

Additional aspects of the present disclosure relate to an oligonucleotide or polynucleotide comprising or incorporating a nucleotide described herein. In some embodiments, the oligonucleotide or polynucleotide is hybridized to at least a portion of a target polynucleotide. In some embodiments, the target polynucleotide is immobilized on a solid support. In some further embodiments, the solid support comprises an array or a plurality of different immobilized target polynucleotides. In further embodiments, the solid support comprises a patterned flow cell. In further embodiments, the patterned flow cell comprises a plurality of nanowells. In further embodiments, the solid support comprises at least 5,000,000 spatially distinguishable sites/cm² that comprise multiple copies of target polynucleotides.

### Methods of Sequencing

Nucleotides comprising a dye compound according to the present disclosure may be used in any method of analysis such as method that include detection of a fluorescent label attached to such nucleotide, whether on its own or incorporated into or associated with a larger molecular structure or conjugate. In this context the term “incorporated into a polynucleotide” can mean that the 5′ phosphate is joined in phosphodiester linkage to the 3′ hydroxyl group of a second nucleotide, which may itself form part of a longer polynucleotide chain. The 3′ end of a nucleotide set forth herein may or may not be joined in phosphodiester linkage to the 5′ phosphate of a further nucleotide. Thus, in one non-limiting embodiment, the disclosure provides a method of detecting a labeled nucleotide incorporated into a polynucleotide which comprises: (a) incorporating at least one labeled nucleotide of the disclosure into a polynucleotide and (b) determining the identity of the nucleotide(s) incorporated into the polynucleotide by detecting the fluorescent signal from the dye compound attached to said nucleotide(s).

Some embodiments of the present disclosure relate to a method of determining the sequence of a target single-stranded polynucleotide, comprising: (a) incorporating the nucleotide as described herein into a copy polynucleotide strand complementary to at least a portion of the target polynucleotide strand, wherein the nucleotide has 2′ deoxyribose moiety and a 3′ blocking group; (b) performing one or more fluorescent measurements for determining the identity of the nucleotide incorporated into the copy polynucleotide strand; and (c) removing the 3′ blocking group from the nucleotide incorporated into the copy polynucleotide strand. In some embodiments, the removal of the 3′ blocking group of the incorporated nucleotide is achieved by a tetrazine reagent described herein, when the incorporated nucleotide is a nucleotide of Formula (I) or (Ia) as described herein, or a nucleotide containing an isonitrile moiety in the 3′ blocking group (e.g., a nucleotide of Formula (IV) as described herein). In some embodiments, step (c) also removes the detectable label of the incorporated nucleotide.

Additional embodiments of the present disclosure relate to a method of determining the sequence of a target single-stranded polynucleotide, comprising: (a) incorporating a nucleotide disclosed herein into a copy polynucleotide strand complementary to at least a portion of the target polynucleotide strand, wherein the nucleotide contains a detectable label attached to the nucleobase via a cleavable linker; (b) performing one or more fluorescent measurements for determining the identity of the nucleotide incorporated into the copy polynucleotide strand; and (c) removing the detectable label from the nucleotide incorporated into the copy polynucleotide strand. In some embodiments, the removal of the detectable label is achieved by a tetrazine reagent described herein, when the incorporated nucleotide is a nucleotide containing a strained unsaturated ring or ring system in the cleavable linker (e.g., the cleavable linker contains an unsubstituted or substituted C₇-C₁₄ unsaturated carbocyclic ring or ring system or unsubstituted or substituted 7 to 14 membered unsaturated heterocyclic ring or ring system, each comprising at least one double bond or one triple bond in the ring or ring system), such as the nucleotide of Formula (IIa) or (IIb) as described herein. In some other embodiments, when the incorporated nucleotide is a nucleotide containing an unsubstituted or substituted tetrazine moiety in the cleavable linker (e.g., a nucleotide of Formula (III) as described herein), the removal of the detectable label is achieved by a cleavage reagent having an unsubstituted or substituted C₇-C₁₄ unsaturated carbocyclic ring or ring system or unsubstituted or substituted 7 to 14 membered unsaturated heterocyclic ring or ring system, each comprising at least one double bond or one triple bond in the ring or ring system. In some embodiments the cleavage reagent is

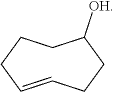

In other embodiments, the removing of detectable label is achieved by an isonitrile cleavage reagent

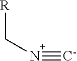

or a salt thereof, wherein R is H, halo, cyano, nitro, an unsubstituted or substituted C₁-C₆ alkyl, C₁-C₆ haloalkyl, unsubstituted or substituted C₁-C₆ alkoxy, C₁-C₆ haloalkoxy, unsubstituted or substituted C₂-C₆ alkenyl, unsubstituted or substituted C₆-C₁₀ aryl, unsubstituted or substituted 5 to 10 membered heteroaryl, unsubstituted or substituted C₃-C₁₀ carbocyclyl, or unsubstituted or substituted 3 to 10 membered heterocyclyl. In some further embodiments, step (c) also removes the detectable label of the incorporated nucleotide.

Some further embodiments of the present application are directed to a method for determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one more of four different types of nucleotides (e.g., dATP, dGTP, dCTP and dTTP or dUTP), under conditions suitable for DNA polymerase-mediated primer extension, and incorporating one type of nucleotides into the sequencing primers to produce extended copy polynucleotides, wherein at least one type of nucleotide is a nucleotide described herein, and wherein each of the four types of nucleotides comprises a 3′ blocking group;
(c) imaging the solid support and performing one or more fluorescent measurements of the extended copy polynucleotides; and
(d) removing the 3′ blocking group of the incorporated nucleotides. In some embodiments, step (d) also removes the labels of the incorporated nucleotides (if the incorporated nucleotides are labeled). In some such embodiments, the labels and the 3′ blocking groups of the incorporated nucleotides are removed in a single chemical reaction. In some further embodiments, the method may also comprises (e) washing the solid support with an aqueous wash solution (e.g., washing the removed label moiety and the 3′ blocking group away from the extended copy polynucleotides). In some embodiments, steps (b) through (e) are repeated at least 30, 40, 50, 60, 70, 80, 90, 100, 110, 120, 130, 140, 150, 160, 170, 180, 190, 200, 250, or 300 cycles to determine the target polynucleotide sequences. In some embodiments, the four types of nucleotides comprise dATP, dCTP, dGTP and dTTP or dUTP, or non-natural nucleotide analogs thereof. In some embodiments, the sequence determination is conducted after the completion of repeated cycles of the sequencing steps described herein.

In some embodiments, at least one nucleotide is incorporated into a polynucleotide (such as a single stranded primer polynucleotide described herein) in the synthetic step by the action of a polymerase enzyme. However, other methods of joining nucleotides to polynucleotides, such as, for example, chemical oligonucleotide synthesis or ligation of labeled oligonucleotides to unlabeled oligonucleotides, can be used. Therefore, the term “incorporating,” when used in reference to a nucleotide and polynucleotide, can encompass polynucleotide synthesis by chemical methods as well as enzymatic methods.

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxyl group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

A particular embodiment of the disclosure provides use of labeled nucleotides comprising dye moiety according to the disclosure in a polynucleotide sequencing-by-synthesis reaction. Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′ blocking group and fluorescent dye compounds can then be removed (deprotected) (simultaneously or sequentially) to expose the nascent chain for further nucleotide incorporation. Typically, the identity of the incorporated nucleotide will be determined after each incorporation step, but this is not strictly essential. Similarly, U.S. Pat. No. 5,302,509 (which is incorporated herein by reference) discloses a method to sequence polynucleotides immobilized on a solid support.

The method, as exemplified above, utilizes the incorporation of fluorescently labeled, 3′-blocked nucleotides A, G, C, and T into a growing strand complementary to the immobilized polynucleotide, in the presence of DNA polymerase. The polymerase incorporates a base complementary to the target polynucleotide but is prevented from further addition by the 3′-blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′ hydroxyl group that serves as a primer or initiation point for the addition of further nucleotides in the sequencing reaction. The region of the template to be sequenced will overhang this free 3′ hydroxyl group on the complementary strand. The overhanging region of the template to be sequenced may be single stranded but can be double-stranded, provided that a “nick is present” on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxyl group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO0157248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxyl group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments, template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO2005/047301 (incorporated herein by reference). A still further example of solid-supported template polynucleotides is where the template polynucleotides are attached to hydrogel supported upon silica-based or other solid supports, for example, as described in WO00/31148, WO01/01143, WO02/12566, WO03/014392, U.S. Pat. No. 6,465,178 and WO00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO00/06770 and WO 01/57248, each of which is incorporated herein by reference. Although one use of the labeled nucleotides of the disclosure is in sequencing-by-synthesis reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotides labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

Thus, the present disclosure also encompasses nucleotides labeled with dye compounds which are dideoxynucleotides lacking hydroxyl groups at both of the 3′ and 2′ positions, such modified dideoxynucleotides being suitable for use in Sanger type sequencing methods and the like.

Nucleotides labeled with dye compounds of the present disclosure incorporating 3′ blocking groups, it will be recognized, may also be of utility in Sanger methods and related protocols since the same effect achieved by using dideoxy nucleotides may be achieved by using nucleotides having 3′ OH blocking groups: both prevent incorporation of subsequent nucleotides. Where nucleotides according to the present disclosure, and having a 3′ blocking group are to be used in Sanger-type sequencing methods it will be appreciated that the dye compounds or detectable labels attached to the nucleotides need not be connected via cleavable linkers, since in each instance where a labeled nucleotide of the disclosure is incorporated; no nucleotides need to be subsequently incorporated and thus the label need not be removed from the nucleotide.

Alternatively, the sequencing methods described herein may also be carried out using unlabeled nucleotides and affinity reagents containing a fluorescent dye. For example, one, two, three or each of the four different types of nucleotides (e.g., dATP, dCTP, dGTP and dTTP or dUTP) in the incorporation mixture of step (a) may be unlabeled. Each of the four types of nucleotides (e.g., dNTPs) has a 3′ blocking group to ensure that only a single base can be added by a polymerase to the 3′ end of the primer polynucleotide. After incorporation of an unlabeled nucleotide in step (b), the remaining unincorporated nucleotides are washed away. An affinity reagent is then introduced that specifically recognizes and binds to the incorporated dNTP to provide a labeled extension product comprising the incorporated dNTP. Uses of unlabeled nucleotides and affinity reagents in sequencing by synthesis have been disclosed in WO 2018/129214 and WO 2020/097607. A modified sequencing method of the present disclosure using unlabeled nucleotides may include the following steps:
(a′) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b′) contacting the solid support with an aqueous solution comprising DNA polymerase and one more of four different types of unlabeled nucleotides (e.g., dATP, dCTP, dGTP, and dTTP or dUTP) under conditions suitable for DNA polymerase-mediated primer extension, and incorporating one type of nucleotides into the sequencing primers to produce extended copy polynucleotides, and wherein each of the four types of nucleotides comprises a 3′ blocking group;
(c′) contacting the extended copy polynucleotides with a set of affinity reagents under conditions wherein one affinity reagent binds specifically to the incorporated unlabeled nucleotides to provide labeled extended copy polynucleotides;
(d′) imaging the solid support and performing one or more fluorescent measurements of the extended copy polynucleotides; and
(e′) removing the 3′ blocking group of the incorporated nucleotides.

In some embodiments of the modified sequencing method described herein, the method further comprises removing the affinity reagents from the incorporated nucleotides. In still further embodiments, the 3′ blocking group and the affinity reagent are removed in the same reaction. In some embodiments, the method further comprises a step (f′) washing the solid support with an aqueous wash solution. In further embodiments, steps (b′) through (f′) are repeated at least 50, 100, 150, 200, 250, 300, 350, 400, 450, or 500 cycles to determine the target polynucleotide sequences. In some embodiments, the set of affinity reagents may comprise a first affinity reagent that binds specifically to the first type of nucleotide, a second affinity reagent that binds specifically to the second type of nucleotide, and a third affinity reagent that binds specifically to the third type of nucleotide. In some further embodiments, each of the first, second and the third affinity reagents comprises a detectable labeled that is spectrally distinguishable. In some embodiments, the affinity reagents may include protein tags, antibodies (including but not limited to binding fragments of antibodies, single chain antibodies, bispecific antibodies, and the like), aptamers, knottins, affimers, or any other known agent that binds an incorporated nucleotide with a suitable specificity and affinity. In one embodiment, at least one affinity reagent is an antibody or a protein tag. In another embodiment, at least one of the first type, the second type, and the third type of affinity reagents is an antibody or a protein tag comprising one or more detectable labels (e.g., multiple copies of the same detectable label).

### EXAMPLES

Additional embodiments are disclosed in further detail in the following examples, which are not in any way intended to limit the scope of the claims.

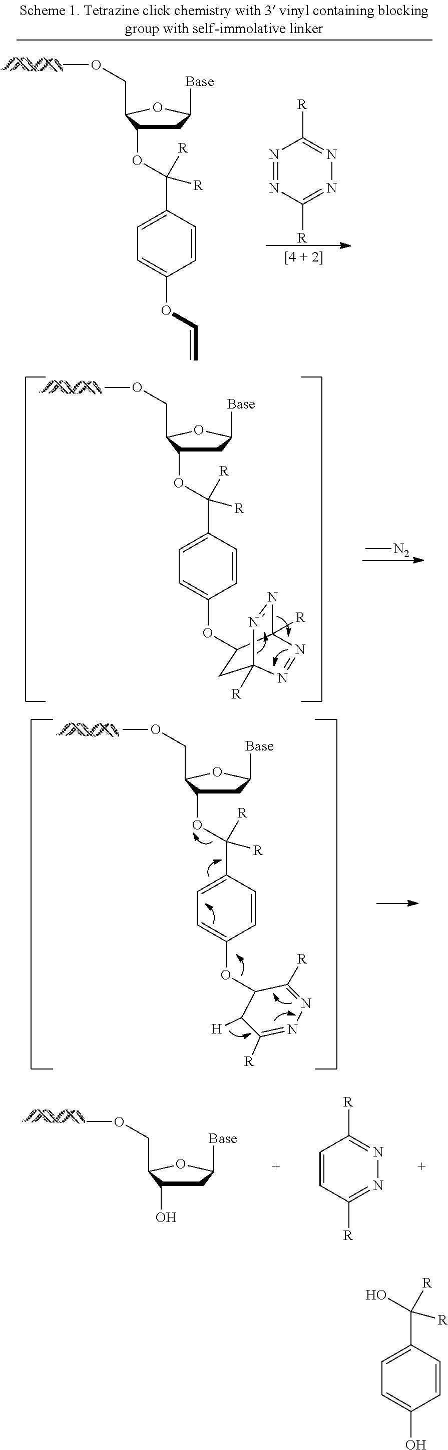

The sequential [4+2]/[4+2] cycloreversion results in the extrusion of nitrogen gas and the formation of an unstable dihydropyridazine which after rearomatization was released to recover the desired 3-OH group. The introduction of a self-immolative linker between the 3′-OH and the vinyl group may improve the stability of the 3′ blocking group. The use of benzylic substitution in linker decomposition is discussed within Saneyoshi et al., Development of Bioreduction Labile Protecting Groups for the 2′-Hydroxyl Group of RNA, Org. Lett. 2020, 22, 6006-6009, incorporated herein by reference. The use of tetrazine in the deblocking of a vinyl group substituted on a benzene ring is discussed in Jimenez-Moreno et al, Vinyl Ether/Tetrazine Pair for the Traceless Release of Alcohols in Cells, Angew. Chem. Int. Ed. 2017, 56, 243-247, which is incorporated by reference. Further discussion of tetrazine used with a self-immolative linker is found in Neumann et al., Tetrazine-Responsive Self-immolative Linkers, ChemBioChem 2017, 18, 91-95 and Neumann et al., Tetrazine-mediated bioorthogonal prodrug-prodrug activation, Chem. Sci., 2018, 9, 7198-7203, both of which are incorporated herein by reference.

Scheme 2A and 2B. Trans-Cyclooctene Self-Immolative Linker Cleavage with a Tetrazine Reagent

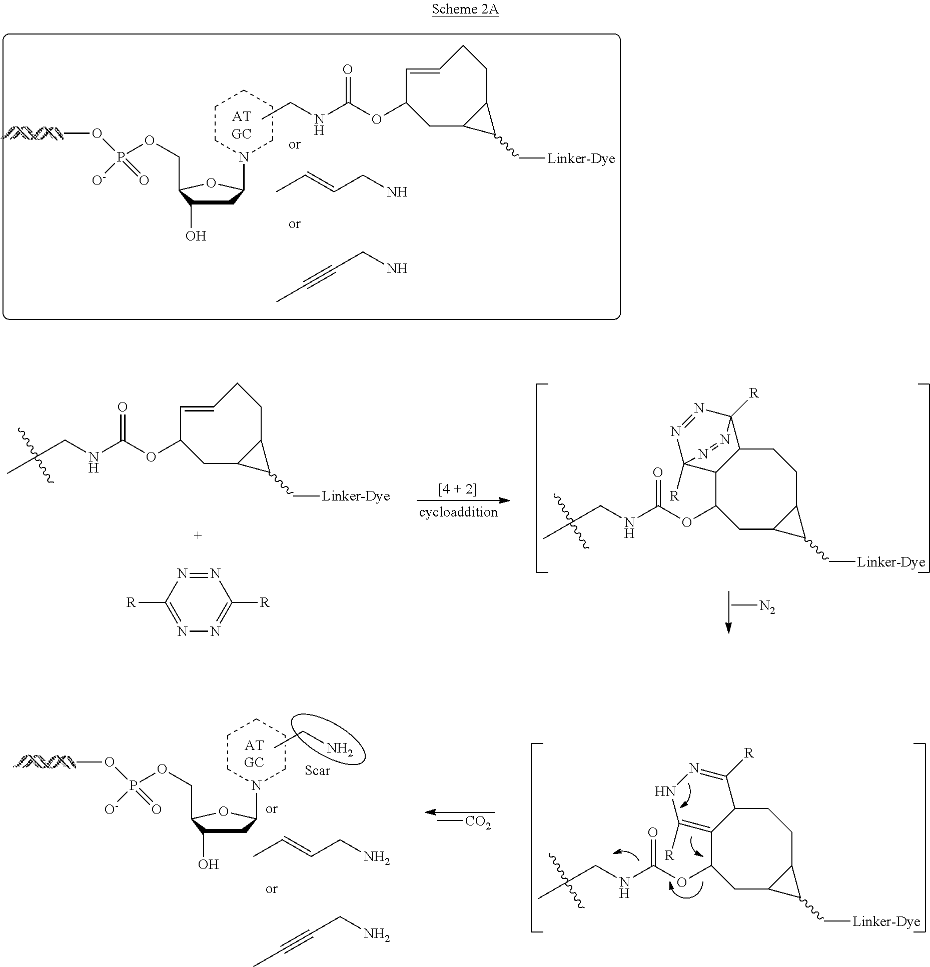

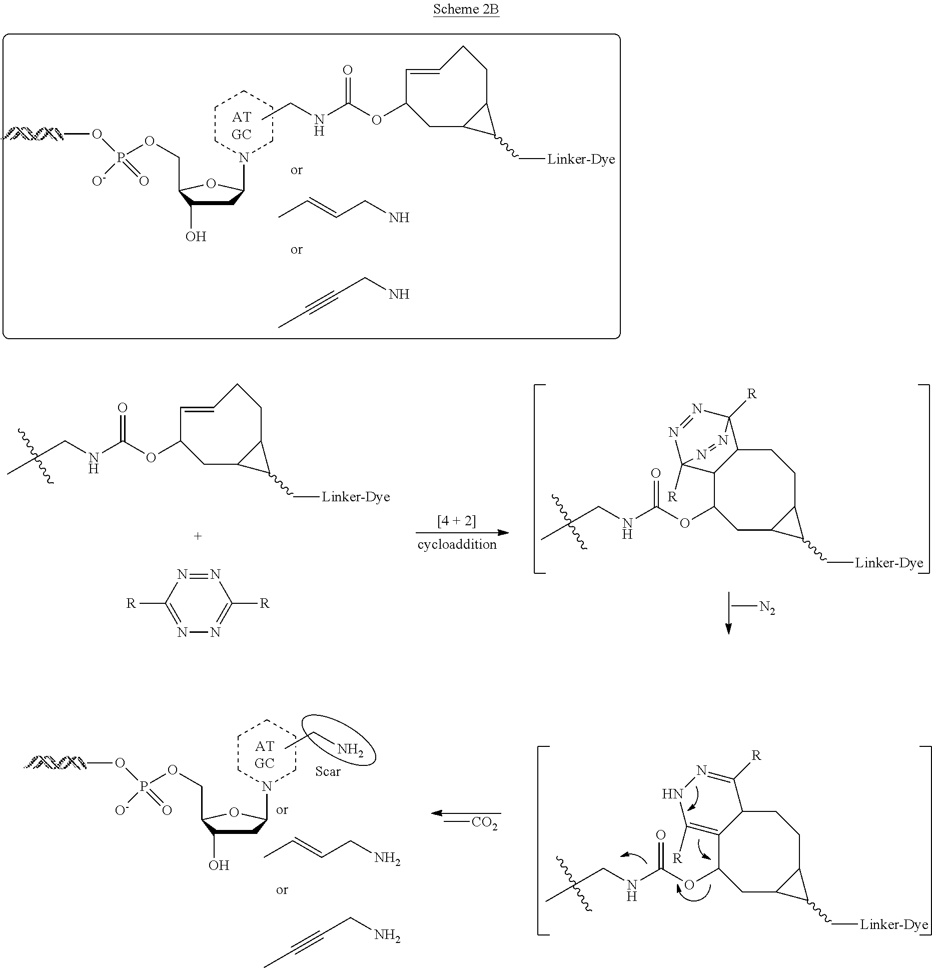

Trans-cyclooctenes are more reactive than traditional alkene and can quickly react with tetrazine to form a ligation product useful for bioconjugation applications. The introduction of an allylic secondary alcohol moiety opens an interesting self-immolative strategy where the ligated product can decompose and sequentially release the linker, carbon dioxide and the amine function attached to the base (e.g., aminomethyl, allylic or propargyl amine, Schemes 2A and 2B). A discussion of tetrazine/trans-cyclooctene click-to-release reactions may be found in Carlson et al., Unraveling Tetrazine-Triggered Bioorthogonal Elimination Enables Chemical Tools for Ultrafast Release and Universal Cleavage, J. Am. Chem. Soc. 2018, 140, 3603-3612 and Rossin et al., Triggered Drug Release from an Antibody-Drug Conjugate Using Fast “Click-to-Release” Chemistry in Mice, Bioconjugate Chem. 2016, 27, 1697-1706, both of which are incorporated by references.

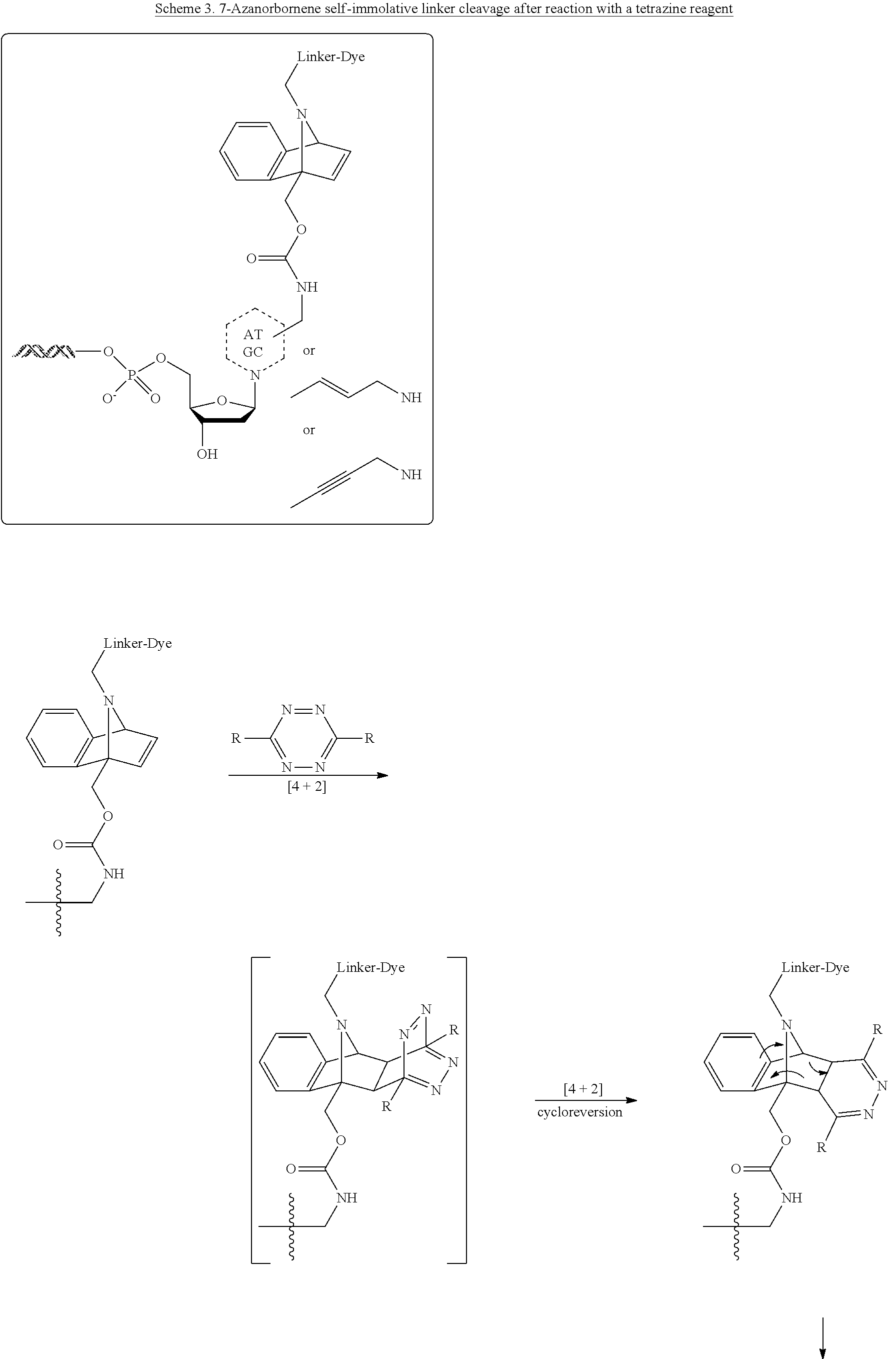

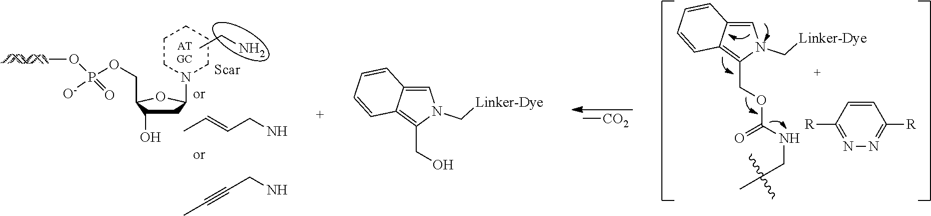

Alternatively, the same carbamate construct could link the base to a 7-azanorbornene where an initial [4+2] would also trigger a series chemical events eventually leading to the decomposition of the self-immolative linker and release of the corresponding amine (Scheme 3). A discussion of a benzonorbornadiene linked to an amine via a carbamate moiety can be found in Xu et al., Rapid and efficient tetrazine-induced drug release from highly stable benzonorbornadiene derivatives, Chem. Commun., 2017, 53, 6271-6274, which is incorporated herein by reference.

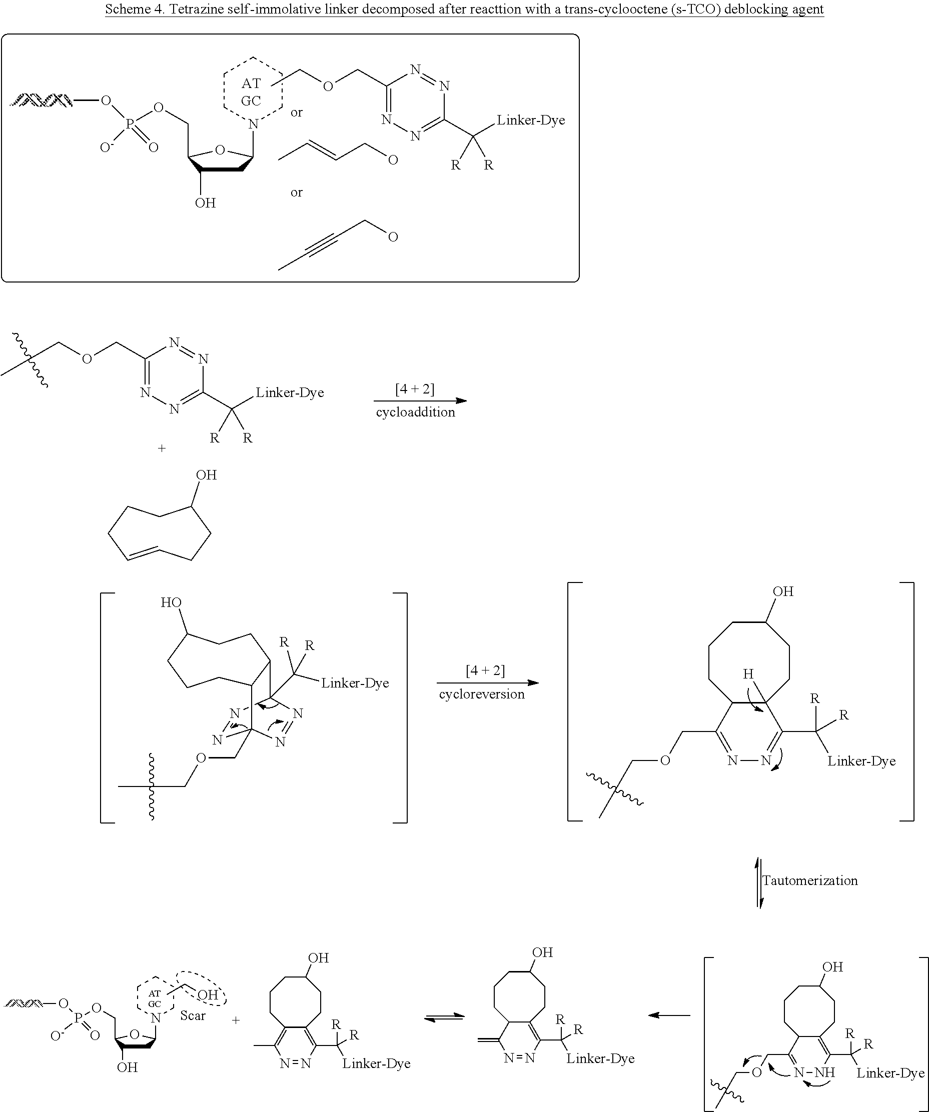

A trans-cyclooctene can be added to react with the tetrazine linker and trigger cascade reactions (i.e., [4+2]/[4+2] cycloreversion, tautomerization, elimination) leading to the decomposition of the self-immolative linker and the release of a “quasi” scarless base (Scheme 4). Zhang et al., Isonitrile induced bioorthogonal activation of fluorophores and mutually orthogonal cleavage in live cells, Chem. Commun., 2022, 58, 573, and discusses the use of tetrazines as self-immolative linkers and is incorporated by reference herein.

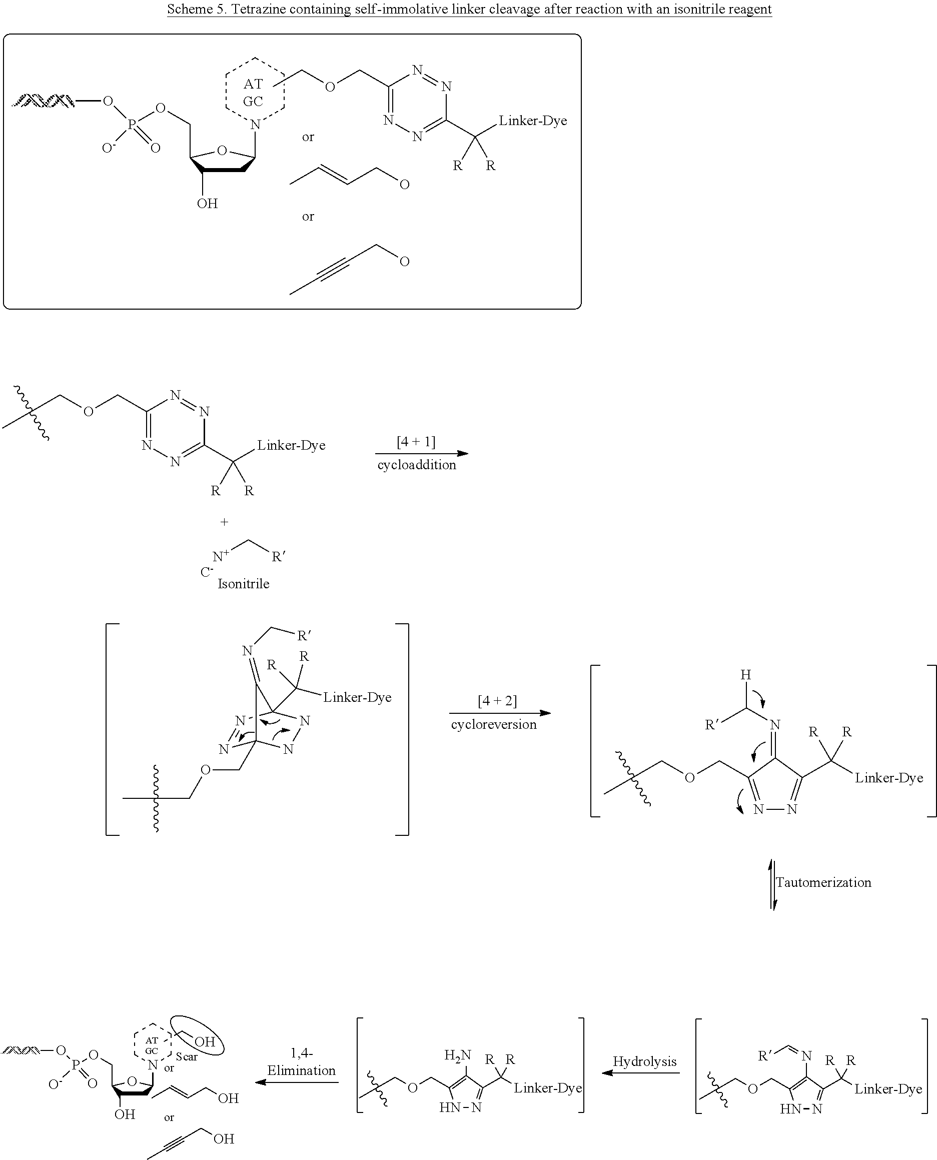

An alternative [4+1] cycloaddition can also be envisioned to form an adduct which after [4+2] cycloreversion can also tautomerize and lead to a hydrolysable imine. The resulting amine can then trigger an elimination step to separate the linker from the alcohol moiety of the nucleobase (Scheme 5). Zhang et al., Isonitrile induced bioorthogonal activation of fluorophores and mutually orthogonal cleavage in live cells, Chem. Commun., 2022, 58, 573, and discusses the use of tetrazines as self-immolative linkers and is incorporated by reference herein.

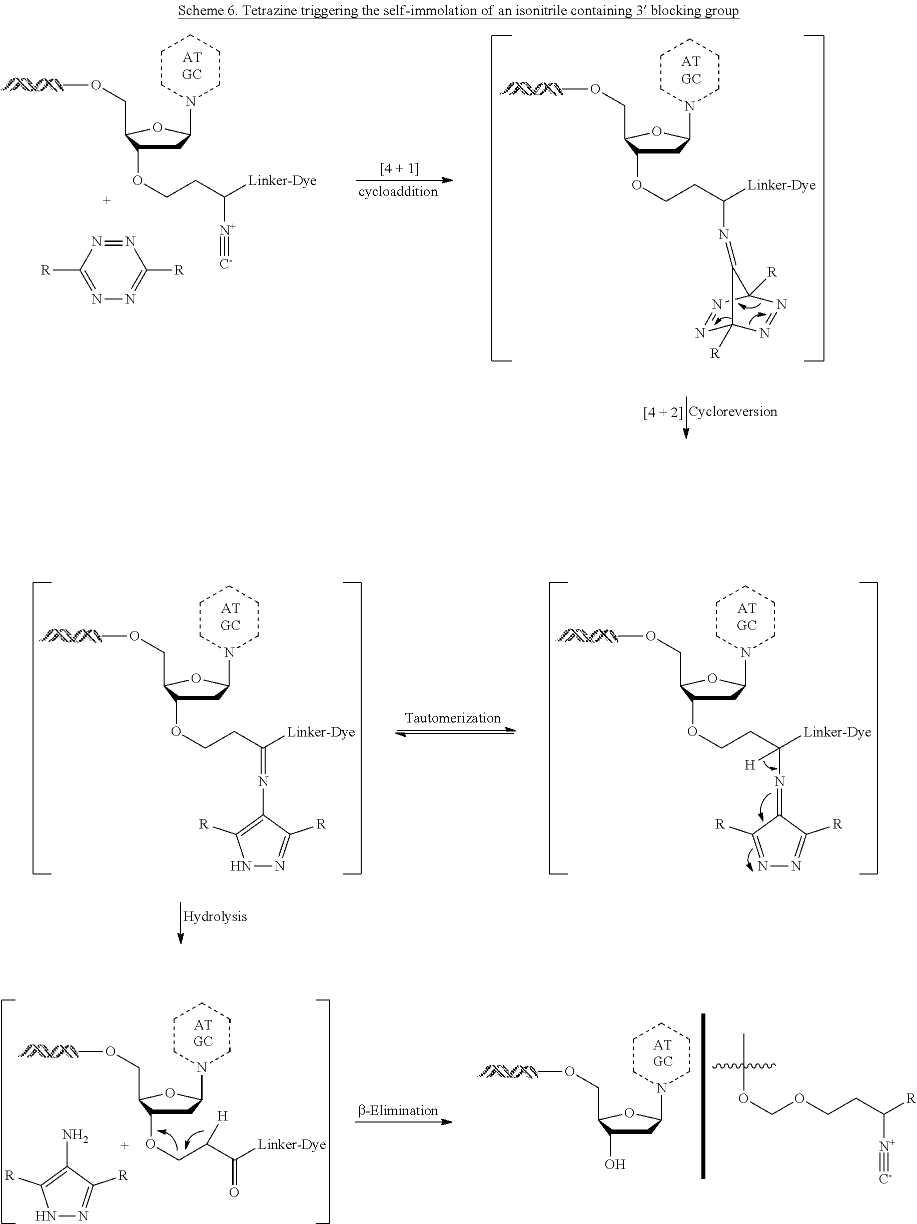

A tetrazine may trigger a self-immolation reaction of an isonitrile-based reversible terminator where the isonitrile group may provide a linkage between a blocking group and a fluorescent dye (Scheme 6). A discussion of tetrazines facilitating isonitrile cleavage reactions can be found in Tu et al., Bioorthogonal Removal of 3-Isocyanopropyl Groups Enables the Controlled Release of Fluorophores and Drugs in Vivo, J. Am. Chem. Soc. 2018, 140, 8410-8414, which is incorporated herein by reference.

1. A nucleoside or nucleotide having the structure of Formula (I): 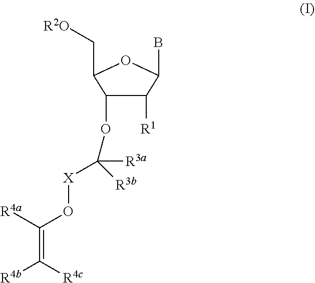
or a salt thereof, wherein:
B comprises a nucleobase;
R¹ is H, hydroxy, halo, unsubstituted or substituted C₁-C₆ alkoxy, or a hydroxy protecting group;
R² is H, a hydroxy protecting group, or —OR² is a monophosphate, diphosphate, triphosphate or phosphorothioate;
X is an unsubstituted or substituted C₆-C₁₀ arylene, or an unsubstituted or substituted 5 to 10 membered heteroarylene;
each of R^{3a} and R^{3b} is independently H, azido, halo, nitro, cyano, unsubstituted or substituted C₁-C₆ alkyl, C₁-C₆ haloalkyl, C₁-C₆ alkoxy, or C₁-C₆ haloalkoxy;
each of R⁴, R^{4b} and R^{4c} is independently H, halo, C₁-C₃ alkyl, C₁-C₃ haloalkyl, C₁-C₃ hydroxyalkyl, azido, cyano, unsubstituted or substituted phenyl, unsubstituted or substituted 5 to 6 membered heteroaryl, unsubstituted or substituted C₃-C₇ cycloalkyl, or unsubstituted or substituted 3 to 7 membered heterocyclyl.

2. The nucleoside or nucleotide of claim 1, wherein the nucleoside or nucleotide has the structure of Formula (Ia): 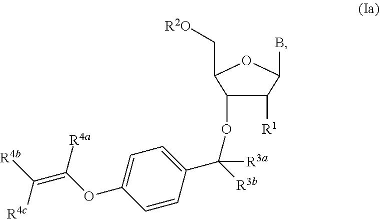
or a salt thereof.

3. The nucleoside or nucleotide of claim 1, wherein at least one of R^{4a}, R^{4b} and R^{4c} is methyl, ethyl, n-propyl, isopropyl, fluoro, chloro, —CHF₂, —CH₂F, —CH₂Cl, —CHCl₂, or —CF₃.

4. The nucleoside or nucleotide of claim 1, wherein each of R^{4a}, R^{4b} and R^{4c} is H.

5. The nucleoside or nucleotide of claim 1, wherein R¹ is H, hydroxy, or a hydroxy protecting group selected from the group consisting of 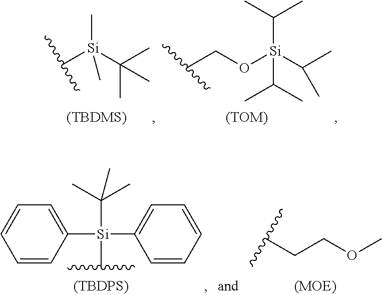
that covalently attached to the 2′ oxygen atom.

6. The nucleoside or nucleotide of claim 1, wherein B comprises a nucleobase covalently bounded to a detectable label, optionally through a linker.

7. The nucleoside or nucleotide of claim 6, B is

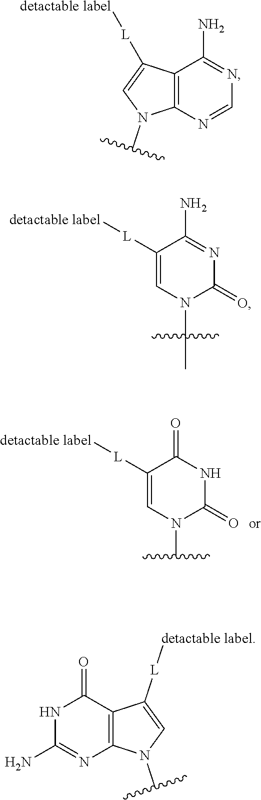

8. The nucleoside or nucleotide of claim 1, wherein the nucleoside or nucleotide is nucleotide having 2′ deoxyribose, and —OR² is triphosphate.

9. An oligonucleotide or polynucleotide comprising a nucleotide of claim 8 incorporated thereof.

10. A method of determining the sequence of a target single-stranded polynucleotide, comprising:
(a) incorporating a nucleotide claim 8 into a copy polynucleotide strand complementary to at least a portion of the target polynucleotide strand;
(b) performing one or more fluorescent measurements for determining the identity of the nucleotide incorporated into the copy polynucleotide strand; and
(c) removing the 3′ blocking group from the nucleotide incorporated into the copy polynucleotide strand.

11. The method of claim 10, wherein the removing of the 3′ blocking group of the incorporated nucleotide is achieved by a tetrazine reagent.

12. A method of controlled synthesis of an oligonucleotide or polynucleotide, comprising:
contacting a nucleotide of claim 8 with the oligonucleotide or polynucleotide in the presence of a polymerase; and
incorporating the nucleotide to the 3′ end of the oligonucleotide or polynucleotide.

13. The method of claim 12, further comprising:
removing the 3′ blocking group of the incorporated nucleotide with a tetrazine reagent to generate a 3′ hydroxy group on the incorporated nucleotide, and
incorporating a second nucleotide.

14. The method of claim 11, wherein the tetrazine reagent is selected from the group consisting of 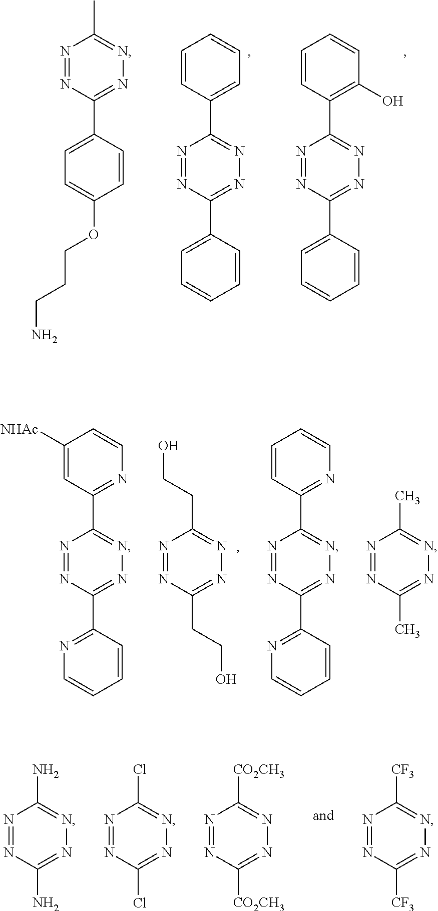
and unsubstituted or substituted variants thereof.

15. A nucleoside or nucleotide comprising a nucleobase attached to a detectable label via a cleavable linker, wherein the nucleoside or nucleotide comprises a ribose or 2′ deoxyribose moiety and a 3′ blocking group, and wherein the cleavable linker comprises an unsubstituted or substituted C₇-C₁₄ unsaturated carbocyclic ring or ring system or unsubstituted or substituted 7 to 14 membered unsaturated heterocyclic ring or ring system, each comprising at least one double bond or one triple bond in the ring or ring system.

16.-24. (canceled)

25. A method of determining the sequence of a target single-stranded polynucleotide, comprising:
(a) incorporating a nucleotide of claim 15 into a copy polynucleotide strand complementary to at least a portion of the target polynucleotide strand;
(b) performing one or more fluorescent measurements for determining the identity of the nucleotide incorporated into the copy polynucleotide strand; and
(c) removing the detectable label from the nucleotide incorporated into the copy polynucleotide strand.

26. (canceled)

27. (canceled)

28. A method of controlled synthesis of an oligonucleotide or polynucleotide, comprising:
contacting a nucleotide of claim 15 with the oligonucleotide or polynucleotide in the presence of a polymerase; and
incorporating the nucleotide to the 3′ end of the oligonucleotide or polynucleotide.

29. (canceled)

30. A nucleoside or nucleotide comprising a nucleobase attached to a detectable label via a cleavable linker, wherein the nucleoside or nucleotide comprises a ribose or 2′ deoxyribose moiety and a 3′ blocking group, and wherein the cleavable linker comprises an unsubstituted or substituted tetrazine moiety.

31.-37. (canceled)

38. A method of determining the sequence of a target single-stranded polynucleotide, comprising:
(a) incorporating a nucleotide of claim 30 into a copy polynucleotide strand complementary to at least a portion of the target polynucleotide strand;
(b) performing one or more fluorescent measurements for determining the identity of the nucleotide incorporated into the copy polynucleotide strand; and
(c) removing the detectable label of the nucleotide incorporated into the copy polynucleotide strand.

39. (canceled)

40. (canceled)

41. (canceled)

42. A method of controlled synthesis of an oligonucleotide or polynucleotide, comprising:
contacting a nucleotide of claim 30 with the oligonucleotide or polynucleotide in the presence of a polymerase; and
incorporating the nucleotide to the 3′ end of the oligonucleotide or polynucleotide.

43. (canceled)

44. A nucleoside or nucleotide comprising a ribose or 2′ deoxyribose moiety and a 3′ blocking group, and a detectable label attached to the 3′ blocking group optionally via a linker, wherein the 3′ blocking group comprises an isonitrile moiety.

45.-50. (canceled)

51. A method of determining the sequence of a target single-stranded polynucleotide, comprising:
(a) incorporating a nucleotide of claim 44 into a copy polynucleotide strand complementary to at least a portion of the target polynucleotide strand;
(b) performing one or more fluorescent measurements for determining the identity of the nucleotide incorporated into the copy polynucleotide strand; and
(c) removing the 3′ blocking group from the nucleotide incorporated into the copy polynucleotide strand.

52. (canceled)

53. A method of controlled synthesis of an oligonucleotide or polynucleotide, comprising:
contacting a nucleotide of claim 44 with the oligonucleotide or polynucleotide in the presence of a polymerase; and
incorporating the nucleotide to the 3′ end of the oligonucleotide or polynucleotide.

54. (canceled)

55. (canceled)
